(19) United States
(12) Patent Application Publication
(10) Pub. No.: US 2010/0185228 A1
(43) Pub. Date: Jul. 22, 2010

(54) VARIABLE STIFFNESS OCCLUDING DEVICE

(76) Inventors: Kurt J. Tekulve, Ellettsville, IN (US); James M. Carlson, Bloomington, IN (US)

Correspondence Address: BRINKS HOFER GILSON & LIONE/CHICAGO/COOK, PO BOX 10395, CHICAGO, IL, 60610, US

(21) Appl. No.: 12/528,922

(22) PCT Filed: Feb. 27, 2008
(86) PCT No.: PCT/US08/02597

§ 371 Date: Apr. 1, 2010

Related U.S. Application Data
(60) Provisional application No. 60/903,707, filed on Feb. 27, 2007.

(51) Int. Cl. A61F 2/01; A61M 29/00

(52) U.S. Cl. 606/200; 606/191

(57) ABSTRACT

An occluding device designed for occlusion of fluid flow through a body cavity. The device comprises a coil and fibers attached to the coil. The coil has a proximal and distal portion, with variable rigidity along the length of the coil. The distal portion has greater rigidity than the proximal portion. The fibers extend from the coil at a length.

### CROSS REFERENCE TO RELATED APPLICATIONS

This application claims priority to PCT patent application PCT/US2008/002597 filed Feb. 27, 2008 and also claims the benefit of U.S. Provisional Application Ser. No. 60/903,707, filed on Feb. 27, 2007, entitled “VARIABLE STIFFNESS OCCLUDING DEVICE,” the entire contents of which are incorporated herein by reference.

### BACKGROUND OF THE INVENTION

The present invention relates to medical devices. More particularly, the invention relates to variable stiffness occluding devices and methods of occluding fluid flow through a body vessel.

Pushable fibered coils have been used as a primary occluding device for treatment of various arteriovenous malformations (AVM) and varicoceles, as well as for many other arteriovenous abnormalities in the body. Occluding devices are also used to repair abnormal shunts between arteries and veins, prevent or reduce blood flow to tumors, stop hemorrhaging as a result of trauma, and stabilize aneurysms to prevent rupture. Pushable fibered coils may be configured in a variety of sizes with varying diameters and may be made of several different materials including stainless steel and platinum. Occlusion devices may vary for differing purposes, e.g., to hold the device in place within a cavity or vessel and to pack the device within the vessel for enhanced occlusion.

Although current pushable fibered coils are adequate, such coils may be improved for more effective occlusion of fluid flow through a lumen of a body vessel. Many medical procedures for occluding blood flow through an artery or vein require a number of coils, since a single coil or two may not be sufficient to effectively occlude blood flow through a lumen of an artery or vein. In many current procedures, many coils may be packed within each other to produce effective cross sectional occlusion of fluid flow through a body vessel. In some instances, these procedures may involve an undesirable amount of additional time and costs.

### BRIEF SUMMARY OF THE INVENTION

The present invention provides an improved occluding device and an improved method of occluding fluid flow through a body cavity. In some embodiments of the present invention, the occluding device comprises a primary coil and a secondary coil. The primary coil is configured to have variable stiffness or rigidity along a proximal and distal portion. A higher first initial tension at the distal portion creates greater rigidity than a comparatively lower second initial tension at the proximal portion. Greater rigidity at the distal portion facilitates placement and positioning of the occluding device in the lumen of a body vessel, and enables the occluding device to stay stationary by retaining its position along an inner wall of the body vessel. The lower second initial tension creates less rigidity in the proximal portion of the primary coil and serves to fill or pack the lumen of the body vessel. Additionally, the occluding device has fibers attached thereto along the length of the primary coil. The fibers include strands comprising of a synthetic polymer such as DACRON™ or another type of a polyester textile fiber. The primary coil is formed into the secondary coil having a series of loops axially spaced apart.

The present invention further includes an embolization kit for occluding fluid flow through a body vessel. The kit comprises a guide catheter and a microcatheter having a proximal end and a distal end. The microcatheter is configured to be passed through the guide catheter to position the microcatheter in the body vessel. The microcatheter has a hub adjacent the proximal end. The kit further includes the occluding device that is introducible through the microcatheter.

Further objects, features, and advantages of the present invention will become apparent from consideration of the following description and the appended claims when taken in connection with the accompanying drawings.

### BRIEF DESCRIPTION OF THE DRAWINGS

FIG. 1 is a cross-sectional environmental view of an occluding device deployed in a body vessel;

FIG. 2a is a side view of an occluding device in accordance with one embodiment of the present invention;

FIG. 2b is a cross-sectional view of the occluding device in FIG. 2a taken along line 2-2;

FIG. 3a is a side view of the primary coil in FIG. 1, in accordance with one embodiment of the present invention;

FIG. 3b is a cross-sectional view of the primary coil in FIG. 3a taken along line 3-3;

FIG. 4 is an end view of the occluding device;

FIG. 5a is an exploded view of an embolization kit for the occluding device in accordance with one embodiment of the present invention;

FIG. 5b is a side view of the embolization kit;

FIG. 6 is a side view of an occluding device in accordance with another embodiment of the present invention;

FIG. 7 is a side view of an occluding device in accordance with yet another embodiment of the present invention;

FIG. 8 is a flowchart of a push embolization method in accordance with one example of the present invention; and

FIG. 9 is a flowchart of a squirt embolization method in accordance with one example of the present invention.

### DETAILED DESCRIPTION OF THE INVENTION

The following provides a detailed description of currently preferred embodiments of the present invention. The description is not intended to limit the invention in any manner, but rather serves to enable those skilled in the art to make and use the invention.

The present invention generally provides an occluding device used for transcatheter embolization and having variable rigidity to provide better attachment of the device to an inner wall of a body vessel and improved occlusion of fluid flow through the vessel. The occluding device is preferably used to occlude fluid flow through a body vessel due to a blood vessel malformation occurring in the brain, like aneurysms, or another part of the body. The occluding device comprises a primary coil having variable rigidity along a proximal portion and a distal portion. In one embodiment, the distal portion has greater rigidity than the proximal portion. Preferably, the primary coil is formed in a helical shape and further defines a secondary coil. To further facilitate occlusion of fluid flow the occluding device comprises fibers attached between loops of the primary coil and extending thereform.

The occluding device also may be used for treatment of renal arteriovenous malfunction (AVM), pulmonary AVM, vascular tumors, low-flow fistulas, trauma related hemorrhages, and visceral vasculature defects including varicoceles, aneurysms, and selected telangiectasias. For example, treatment of visceral vasculature defects may include but are not limited to embolotherapy on gastroduogenal hemorrhages, hepatic aneurysms, celiac aneurysms, internal iliac aneurysms, and internal spermatic varicoceles.

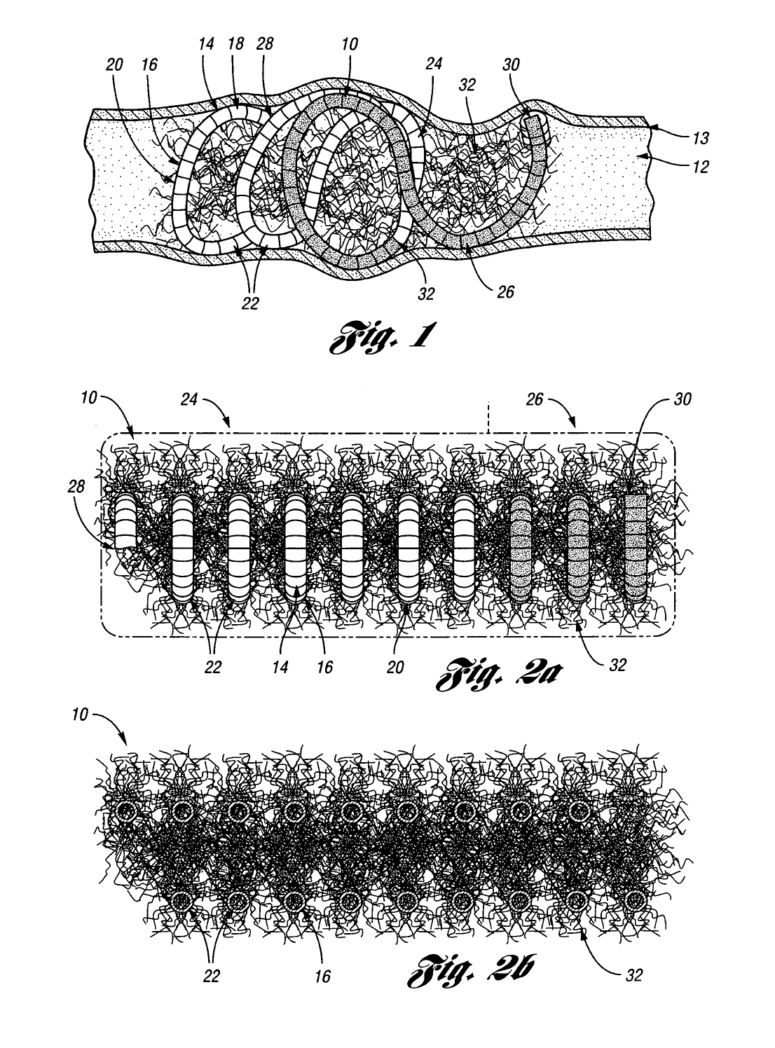

FIG. 1 illustrates an occluding device 10 in a deployed state for occlusion of fluid flow through a lumen of a body vessel 12 in accordance with one embodiment of the present invention. As shown in FIG. 1, the occluding device 10 is positioned to engage an inner wall 13 of the body vessel 12 and comprises a primary coil 14 and a secondary coil 16. Preferably, the primary coil 14 comprises a primary body 18 that has a helical shape and forms the secondary coil 16. The secondary coil 16 comprises a secondary body 20 that forms a series of loops 22. The series of loops 22 define a cross-sectional area formed axially along the secondary coil 16.

As shown in FIGS. 1-2b, the primary coil 14 further comprises a proximal portion 24 and a distal portion 26. The proximal portion 24 extends from a proximal end 28 to the distal portion 26 which, in turn, extends to a distal end 30 of the occluding device 10. The proximal portion 24 integrally abuts the distal portion 26 at a connecting point 32. Along the length of the primary coil 14 there is a difference in rigidity between the portions of the occluding device 10 at the connecting point 32. In this embodiment, the distal portion 26 is more rigid than the proximal portion 24 to facilitate placement of the occluding device 10 in the body cavity 12 and to prevent the occluding device 10 from migration by retaining its position along the inner wall 13 of the body vessel 12. The proximal portion 24 is less rigid and serves to pack in the more rigid distal portion 26 inside the lumen of the body cavity 12. Preferably, to assist in occluding fluid flow through the lumen of the body vessel 12, the proximal portion 24 and the distal portion 26 of the primary coil 14 further include a series of fibers 34 attached between loops of the primary coil and extending therefrom.

In this embodiment of the present invention, the distal portion 26 has a first initial tension and the proximal portion 24 has a second initial tension. The second initial tension is less than the first initial tension. Consequently, along the length of the primary coil 14, a difference in initial tension between the portions of the occluding device 10 is at the connecting point 32. In this embodiment, the proximal portion has an initial tension of between about 5 to 60 grams of weight, and preferably between about 10 to 30 grams of weight. The distal portion preferably has an initial tension of between about 65 to 120 grams of weight, and preferably between about 75 to 100 grams of weight. Initial tension may be defined to be the amount of force required to cause a 4 centimeter length of coil to begin to elongate. The initial tension may also be defined by the amount of force required to cause a coil to begin elongating at a ratio of between about 1.25 to 15 grams per centimeter, and preferably between about 2.5 to 7.5 grams per centimeter. Without limiting the invention, it is believed that the initial tension of the distal portion provides rigid support thereto to minimize migration of the device within the lumen and that the initial tension of the proximal portion provides the occluding device the capability of being folded across the diameter of the distal portion within the lumen of a body vessel after deployment from a catheter.

As shown in FIGS. 2a and 2b, the occluding device 10 comprises the primary coil 14 formed to define the secondary coil 16. The primary coil 14 has a helical shape that forms the primary body 18 and comprises of the proximal and distal portions. The secondary coil 16 comprises the secondary body 20 and forms the series of loops 22. Preferably, the secondary body 20 has a length of between about 2 to 30 centimeters. As shown in FIG. 2a, the series of loops 22 define a cross-sectional lumen formed axially along the length of the secondary coil 16 and is preferably spaced apart by up to about 5 millimeters of curl space. Preferably, the occluding device 10 further includes the fibers 34 wedged or attached to the primary coil 14 and extending thereform. In this embodiment, as shown in FIG. 2a, the series of loops 22 along the secondary coil 16 has a uniform curl space and outside diameter between each loop.

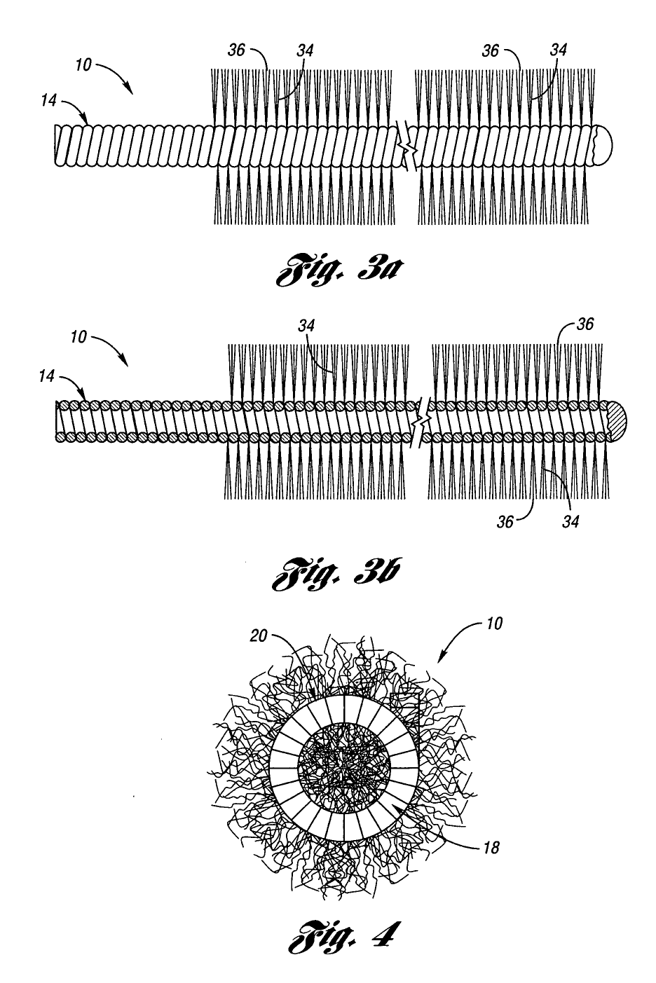

As shown in FIGS. 3a and 3b, the fibers 34 are spaced apart from each other and are held between helical loops of the primary coil 14. Preferably, the fibers 34 include strands 36 comprising of a synthetic polymer such as a polyester textile fiber, e.g., DACRON™. As desired, the strands may be held between adjacent loops, alternating loops, alternating double loops, or any desired configuration.

Preferably, the strands 36 have a length extending from the primary coil 14 of between 3 to 8 millimeters, and preferably between about 5 to 6 millimeters as desired. In this embodiment, the fibers are spaced apart from each other by about 1 to 2 millimeters. Preferably, the strands 28 have an outer diameter of about 0.00050 to 0.00100 inch.

FIG. 4 illustrates an end view of the occluding device 10. The secondary coil 16 may have an outside diameter ranging from about 3 to 15 millimeters. In accordance with one embodiment, the distal portion 26 of the occluding device 10 may have a greater outside diameter than the proximal portion 24. Preferably, the outside diameter of the distal portion 26 establishes greater rigidity, which facilitates retention of the device along the inner wall of the body vessel 12. The proximal portion 24 may have a variable outside diameter along the length of the secondary coil 16, creating the series of loops 22 with variable diameter.

In one embodiment, the occluding device 10 may comprise of at least one or more metals and metal alloys to create variable rigidity along the length of the primary coil 14. In this embodiment, the primary coil 14 may comprise platinum and platinum alloys. In another embodiment, the distal portion may be comprised generally of palladium and the proximal portion may comprise a less rigid alloy, e.g., palladium alloy.

In another embodiment, the distal portion 26 of the primary coil 14 may have a tensile strength of between about 200,000 and 400,000 pounds per square inch and the proximal portion may have a tensile strength of between about 50,000 and 350,000 pounds per square inch. It has been determined that the tensile strength range described above provides the proximal portion with the capability of being flexible, malleable, and folded. Furthermore, the tensile strength differential between the proximal portion 24 and the distal portion 26 of the occluding device 10 facilitates occlusion of fluid through the body cavity 12.

At least part of the device 10 may be made of any suitable material including, in one embodiment, a superelastic material, stainless steel wire, cobalt-chromium-nickel-molybdenum-iron alloy, or cobalt-chrome alloy. It is understood that the device 10 may also be formed of any suitable material that will result in a self-opening or self-expanding device 10, such as shape memory materials. Shape memory materials or alloys have the desirable property of becoming rigid, i.e., returning to a remembered state, when heated above a transition temperature. A shape memory alloy suitable for the present invention is Ni—Ti available under the more commonly known name Nitinol. When this material is heated above the transition temperature, the material undergoes a phase transformation from martensite to austenic, such that the material returns to its remembered state. The transition temperature is dependent on the relative proportions of the alloying elements Ni and Ti and the optional inclusion of alloying additives.

In one example, the device 10 may be made of Nitinol with a transition temperature that is slightly below normal body temperature of humans, which is about 98.6° F. Thus, when the device 10 is deployed in a body vessel and exposed to normal body temperature, the alloy of the device 10 will transform to austenite, that is, the remembered state, which for one embodiment of the present invention is the expanded state when the device 10 is deployed in the body vessel. To remove the device 10 it is cooled to transform the material to martensite which is more ductile than austenite, making the device 10 more malleable. As such, the device 10 can be more easily collapsed and pulled into a lumen of a catheter for removal.

In another example, the device 10 may be made of Nitinol with a transition temperature that is above normal body temperature of humans, which is about 98.6° F. Thus, when the device 10 is deployed in a body vessel and exposed to normal body temperature, the device 10 is in the martensitic state so that the device 10 is sufficiently ductile to bend or form into a desired shape. To remove the device 10, the device 10 is heated to transform the alloy to austenite so that it becomes rigid and returns to a remembered state.

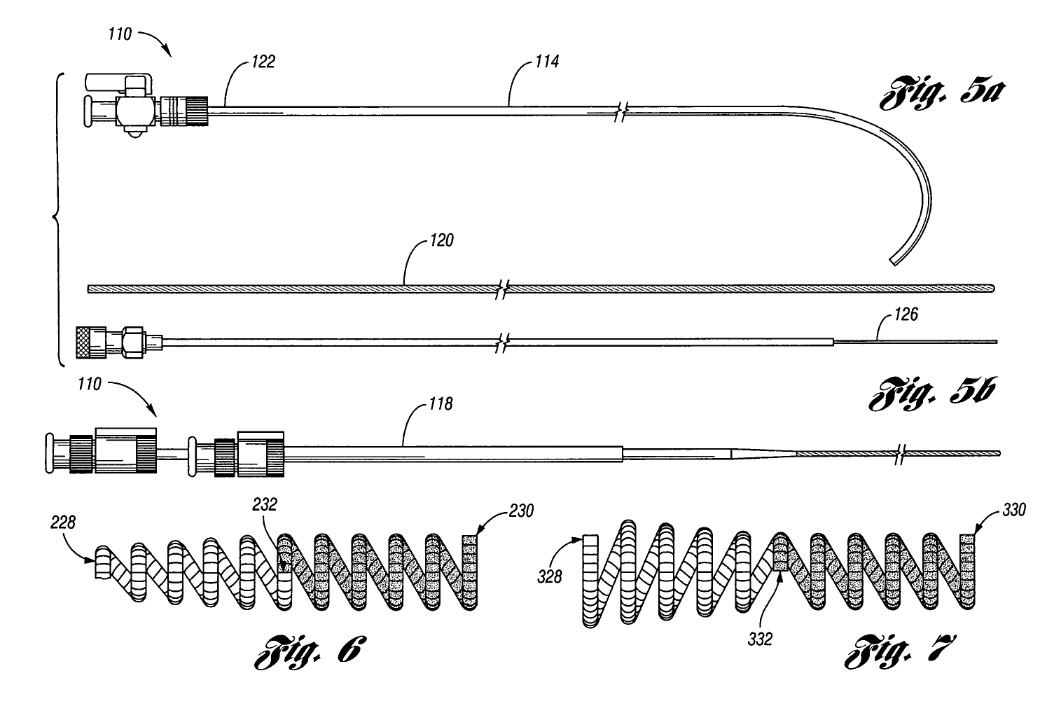

FIGS. 5a and 5b illustrate a body cavity embolization kit 110 which implements the occluding device in accordance with one embodiment of the present invention. As shown, the kit 110 includes a microcatheter 114 preferably made from a soft, flexible material such as silicone or any other suitable material. Generally, the microcatheter 114 has a proximal end 122, a distal end 124, and a plastic adapter or hub 116 to receive apparatus to be advanced therethrough. In this embodiment, the inside diameter of the microcatheter may range between 0.014 and 0.027 inch. The kit 110 further includes a guide wire 120 which provides the guide catheter 118 a path during insertion of the guide catheter 118 within a body cavity. The size of the wire guide is based on the inside diameter of the guide catheter.

In this embodiment, the kit 110 further includes a polytetrafluoroethylene (PTFE) guide catheter or sheath 118 for percutaneously introducing the microcatheter 114 in a body cavity. Of course, any other suitable material may be used without falling beyond the scope or spirit of the present invention. The guide catheter 118 may have a size of about 4-French to 8-French and allows the microcatheter 114 to be inserted therethrough to a desired location in the body cavity. The guide catheter 118 receives the microcatheter 114 and provides stability of the microcatheter 114 at a desired location of the body cavity. For example, the guide catheter 118 may stay stationary within a common visceral artery, e.g., a common hepatic artery, and add stability to the microcatheter 114 as the microcatheter is advanced through the guide catheter to a point of occlusion in a connecting artery, e.g., the left or right hepatic artery.

When the distal end 124 of the microcatheter 114 is at the point of occlusion in the body cavity, the occluding device is loaded at the proximal end 122 of the microcatheter 114 and is advanced through the microcatheter for deployment through the distal end 124. In this embodiment, a push wire 126 is used to mechanically advance or push the occluding device through the microcatheter 114. The size of the push wire used depends on the diameters of the microcatheter. As mentioned above, the distal portion 26 serves to hold the coil in place along the inner wall of the body cavity 13. The proximal portion 24 and fibers 34 serve to occlude fluid passage by filling the lumen of the body cavity 12.

It is to be understood that the body cavity embolization kit 110 described above is merely one example of a kit that may be used to deploy the occluding device in a body vessel. Of course, other kits, assemblies, and systems may be used to deploy any embodiment of the occluding device without falling beyond the scope or spirit of the present invention.

FIG. 6 illustrates an occluding device 210 in accordance with another embodiment of the present invention. As shown, the device 210 includes a proximal portion 224 of a secondary coil 216 having a variable outside diameter. In this embodiment, the secondary coil 216 proximally tapers from the connecting point 232 to the proximal end 228 of the proximal portion 224. Preferably, the proximal portion 224 has a relatively larger outside diameter than the outside diameter of the distal portion 226 of the secondary coil 216. The variable outside diameter of the proximal portion of the secondary coil 216 provides further enhanced packing of the device 210 for enhanced occlusion.

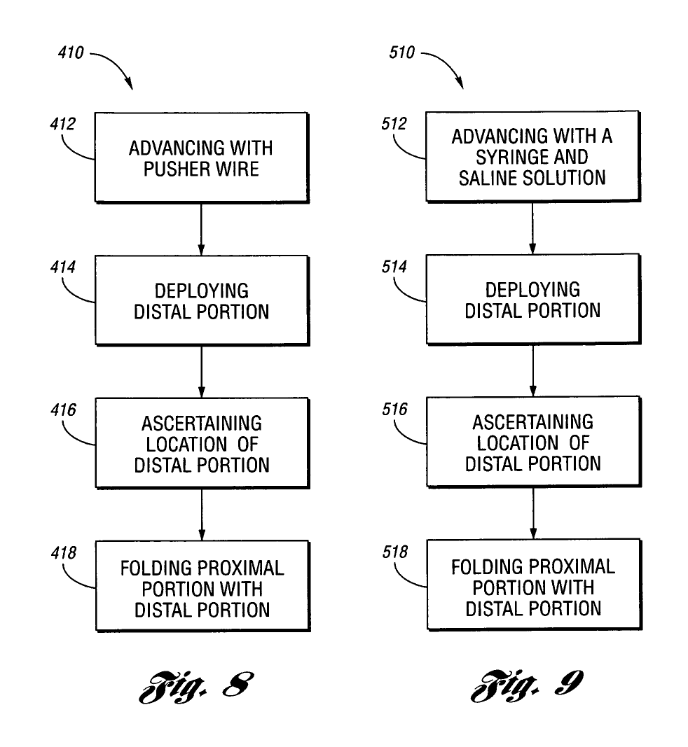

FIG. 7 illustrates another occluding device 310 in accordance with yet another embodiment of the present invention. As shown, the device 310 includes a proximal portion 324 of a secondary coil 316 having a variable outside diameter. In this embodiment, the secondary coil 316 proximally flares from the connecting point 332 to the proximal end 328 of the proximal portion 324. Preferably, the proximal portion 324 has a relatively larger outside diameter than the outside diameter of the distal portion 326 of the secondary coil 316. The variable outside diameter of the proximal portion of the secondary coil provides further enhanced packing of the device for enhanced occlusion.

The occluding device may be deployed in a body vessel by a push embolization method or a squirt embolization method in accordance with the present invention. FIG. 8 depicts a push embolization method 410 of transcatheter embolization using an embodiment of the occluding device. As typically performed in embolotherapy, an introducer or a guide catheter is percutaneously introduced into the body vessel of a patient and a microcatheter is passed through the guide catheter to position the microcatheter at a desired point of occlusion in the body vessel.

The occluding device, which is elongated to its full length within a cartridge, is loaded in the hub at the proximal end of the microcatheter. In step 412, the device is advanced by the pusher wire in accordance with this method of deploying the occluding device.

In step 414, a first portion of the distal portion of the occluding device, e.g., a first loop of the secondary coil, is deployed at the desired point of occlusion in the body vessel as a remaining portion of the occluding device is held in the microcatheter. The first portion of the coil may be between about 5% to 10% of the length of the coil. The first portion begins to hold the device in place within the vessel and the remainder of the distal portion further enhances this feature. In step 416, the location of the first portion in the body vessel is ascertained by any suitable means, such as by fluoroscopy, relative to the body vessel. When the distal portion is at the desired point of occlusion in the body vessel, the proximal portion is folded across the lumen of the body vessel to pack the coil and occlude the body vessel in step 418. Preferably, the proximal portion is folded within the distal portion by moving the catheter reciprocally back and forth relative to the body vessel as the proximal portion is deployed from the microcatheter. As a length of the proximal portion is being deployed, the distal end of the microcatheter is moved back. The microcatheter is then moved forward against the length of the proximal portion, thereby folding the length of the proximal portion at the desired point of occlusion. The microcatheter is moved back and forth until the proximal portion is folded within the distal portion and the occluding device is in a packed state.

However, if it is ascertained in step 416 that the distal portion of the occluding device is not at the desired point of occlusion, then the position of the microcatheter is moved fore or aft relative to the body vessel such that the distal portion is placed at the desired point of occlusion.

FIG. 9 illustrates a squirt embolization method 510 of transcatheter embolization using an embodiment of the occluding device of the present invention. As typically performed in embolotherapy, a guide catheter is introduced into the body vessel as described above in the push embolization method. Once the microcatheter is passed through the guide catheter and the occluding device is loaded at the hub of the microcatheter, the occluding device is advanced in step 512 through the microcatheter with use of a luer lock syringe and saline solution. In step 514, a first portion of the distal portion, e.g., a first loop of the secondary coil, is deployed at the desired point of occlusion in the body vessel as a remaining portion of the distal portion is held in the microcatheter. The first portion of the coil may be between about 5% to 10% of the length of the coil.

In step 516, the location of the first portion in the body vessel is ascertained by any suitable means, such as by fluoroscopy, relative to the body vessel. If the first portion of the coil is at the desired point of occlusion in the body vessel, then the remaining portion is introduced together with the first portion with the saline solution. The distal portion holds the device in place within the vessel. Then, the proximal portion is packed within the distal portion to occlude the body vessel. Preferably, the proximal portion is folded by moving the distal end of the microcatheter reciprocally back and forth relative to the body vessel as described above to pack the coil and occlude the body vessel. However, if it is ascertained in step 516 that the first portion is not at the desired point of occlusion, then the position of the microcatheter is moved fore or aft relative to the body vessel such that the first loop is placed at the desired point of occlusion.

While the present invention has been described in terms of preferred embodiments, it will be understood, of course, that the invention is not limited thereto since modifications may be made to those skilled in the art, particularly in light of the foregoing teachings.

1. An occluding device for occlusion of a body cavity, the device comprising:
a coil having a proximal portion and a distal portion, the coil having variable rigidity along the proximal and distal portions, the distal portion having a first initial tension and the proximal portion has a second initial tension less than the first initial tension defining the distal portion having a greater rigidity than the proximal portion; and
fibers attached to the coil and extending therefrom.

2. The device of claim 1 wherein the coil is a primary coil formed into a secondary coil having a series of loops axially spaced apart by up to about 5 millimeters curl space.

3. An occluding device for occlusion of a body cavity, the device comprising a primary coil having a proximal portion and a distal portion, the coil having variable rigidity along the proximal and distal portions, at least the distal portion of the primary coil being adapted to take the form in the body cavity of a secondary coil, and fibers attached to the primary coil and extending therefrom, wherein the rigidity of the distal portion of the primary coil is sufficiently high to hold the occluding device in position in the body cavity and wherein the rigidity of the proximal portion of the primary coil is sufficiently low to enable the proximal portion to be folded within the secondary coil formed by the distal portion of the primary coil, thereby with the fibers to occlude the cavity.

4. The device of claim 3, the distal portion having a first initial tension and the proximal portion having a second initial tension less than the first initial tension defining the distal portion having a greater rigidity than the proximal portion.

5. The device of claim 2, wherein the secondary coil has a variable outside diameter, the variable outside diameter being configured to proximally flare or proximally taper along the length of the proximal portion.

6. The device of claim 1, wherein the first initial tension is between about 65 to 120 grams of weight and preferably between about 75 to 100 grams of weight, and the second initial tension is between about 5 to 60 grams of weight and preferably between about 10 to 30 grams of weight.

7. The device of claim 1 wherein the distal portion comprises a first metallic, preferably palladium alloy and the proximal portion comprises a second alloy of the same metal, less rigid than the first alloy.

8. The device of claim 1 wherein the distal portion has a tensile strength of between about 200,000 and 400,000 pounds per square inch and the proximal portion has a tensile strength of between about 50,000 and 350,000 pounds per square inch.

9. The device of claim 1 wherein the coil has a length of between about 3 to 20 centimeters.

10. The device of claim 1 wherein the fibers are made of a synthetic polyester textile fiber.

11. The device of claim 1 wherein the coil is a primary coil formed in a secondary coil having a series of loops, the fibers including strands attached to the series of loops, preferably wherein each of the strands extends from the coil at a length of between about 3 to 8 millimeters and having an outer diameter of about 0.00050 to 0.00100 inch.

12. An embolization kit for occluding fluid flow through a body vessel, the kit comprising:
a guide catheter;
a microcatheter having proximal and distal ends and being configured to be passed through the guide catheter to position the microcatheter in the body vessel, the microcatheter having a hub adjacent the proximal end; and
an occluding device comprising:
a coil having a proximal portion and a distal portion, the coil having variable rigidity along the proximal and distal portions, the distal portion having a greater rigidity than the proximal portion; and
fibers attached to the coil and extending thereform.

13. The embolization kit of claim 12, wherein the guide catheter is between about 4-French and 8-French.

14. The embolization kit of claim 12, further comprising a pusher wire for advancing the occluding device.

15. The embolization kit of claim 12 wherein the first initial tension is between about 65 to 120 grams of weight, and the second initial tension is between about 5 to 60 grams of weight.

16. The embolization kit of claim 12 wherein the first initial tension is between about 75 to 100 grams of weight, and the second initial tension is between about 10 to 30 grams of weight.

17. The embolization kit of claim 12 wherein the distal portion has a tensile strength of between about 200,000 and 400,000 pounds per square inch and the proximal portion has a tensile strength of between about 50,000 and 350,000 pounds Per square inch.

18. The embolization kit of claim 12 wherein the coil has a length of between about 3 to 20 centimeters.

19. A method of occluding fluid flow through a body vessel, the method comprising:
providing an occluding device comprising a coil and fibers attached to the coil and extending therefrom, the coil having a proximal portion and a distal portion, the coil having variable rigidity along the proximal and distal portions, the distal portion having a greater rigidity than the proximal portion;
deploying the distal portion at a desired point of occlusion in the body vessel to hold the device in place within the body vessel; and
deploying the proximal portion across the lumen of the body vessel within the distal portion to pack the coil and occlude the body vessel.
